(19) United States
(12) Patent Application Publication
(10) Pub. No.: US 2013/0137188 A1
(43) Pub. Date: May 30, 2013

(54) METHOD OF DETECTING RUPTURE OF MEMBRANES

(71) Applicants: Thierry Paper, Strasbourg (FR); Françoise Rakotondramanga, Schweighouse Sur Moder (FR)

(72) Inventors: Thierry Paper, Strasbourg (FR); Françoise Rakotondramanga, Schweighouse Sur Moder (FR)

(73) Assignee: BIOSYNEX, Eckbolsheim (FR)

(21) Appl. No.: 13/701,805

(22) PCT Filed: Jun. 1, 2011
(86) PCT No.: PCT/FR11/51254

§ 371 Date: Feb. 14, 2013

(30) Foreign Application Priority Data
Jun. 3, 2010 (FR) 1054374

(51) Int. Cl. G01N 33/68

(52) U.S. Cl. CPC G01N 33/689
USPC 436/501; 422/69

(57) ABSTRACT

The present invention relates to a method of detecting rupture of fetal membranes and to a device for implementing said method. In particular, a method of in-vitro detection of a rupture of fetal membranes comprising a step of simultaneously searching, within a specimen of vaginal or cervical secretions, for the alpha fetal protein (AFP) and for the insulinomimetic growth factor-binding protein 1 (IGFBP-1). The present invention finds, in particular, an application in the medical field, especially in the obstetric field.

### PRIORITY CLAIM

This application is a National Phase entry of PCT Application No. PCT/FR2011/051254, filed Jun. 1, 2011, which claims priority from French Application No. 1054374 filed Jun. 3, 2010, the disclosures of which are hereby incorporated by referenced herein in their entirety.

### TECHNICAL FIELD

The present invention relates to a method of detecting rupture of fetal membranes and to a device for implementing said method.

The present invention finds in particular an application in the medical field, the diagnostic field, in particular in the obstetric field.

In the description below, the references between square brackets ([ ]) refer to the list of references provided at the end of the text.

### BACKGROUND OF THE INVENTION

During pregnancy, women may be subjected to various factors and/or events that can endanger the pregnancy. The rupture of fetal membranes is one of these events, particularly when the rupture of the membranes is premature.

The origin of this rupture can vary. It may, for example, be a direct or indirect trauma, such as a blow to the abdomen, a circulatory event, sexual relations, etc. It may also be due to an endogenous membrane fragility (collagen deficiency, malnutrition, smoking), a twin pregnancy, hydramnios, a complication related to an amnioscopy, or to the insertion of a laminaria during a termination of pregnancy. It may also be of infectious origin and due to chorioamnionitis, vulvovaginitis or endometritis. Finally, in 60% of cases, the cause of the rupture is unknown.

The rupture of fetal membranes leads to flowing of the amniotic fluid and represents an “open door” for all pathological agents: bacteria, viruses, etc. These agents can infect, for example, the content of the uterus, the fetus.

Moreover, the loss of amniotic fluid leads to a decrease in the space available for the development of the fetus. This decrease can, when the rupture takes place before the period of 24 weeks of amenorrhea, endanger the vital prognosis of the fetus, leading to a miscarriage and/or resulting in the birth of a non-viable baby. After this 24-week period, the vital prognosis of the infant is still in question, resulting in premature labor and/or an infection of the fetus that may result in voluntary inducing of labor. Moreover, the decrease in the volume of amniotic fluid can cause problems of malformation, limb atrophy, and developmental abnormality of various parts of the body.

The diagnosis of the rupture of fetal membranes is essentially clinical. It involves an examination with a speculum via which a flow of amniotic fluid is visualized. This diagnostic test is a test which is not very sensitive and which is not very suitable for the diagnosis of microruptures, which does not make it possible to rapidly and easily detect the rupture of membranes, and which is especially related to the ability of the practitioner to be able to identify/visualize the flow of the fluid.

There are also other tests, for example the nitrazine test or the diamine oxidase test, for detecting a rupture of fetal membranes.

However, these tests are not reliable and can lead to false-negative results (patients for whom the result is a false negative therefore have a pregnancy at risk and are not managed and/or treated) and false-positive results (patients for whom the result is a false positive do not have a pregnancy at risk and may be managed in the same way as a woman with rupture, with in particular the risk of a needless triggering of labor). The nitrazine test, nitrazine being a pH indicator, is subject to interference from numerous sources, in particular interference from blood, sperm, antiseptics or vaginal infections. The diamine oxidase test uses implementation techniques which are based on radioimmunology and are therefore not very practical. Its sensitivity ranges from 84% to 91%. Its specificity is limited by the interference from blood.

Diagnostic tests based on the detection of insulin-like growth factor-binding protein 1 (IGFBP-1) using specific antibodies in a vaginal or cervical specimen have also been developed and marketed. This protein is present in amniotic fluid at a concentration 100 to 1000 times higher than that of maternal serum (Rutanen et al. Radioimmunoassay of placenta protein 12: levels in amniotic fluid, cord blood and serum of healthy adults, pregnant women and patients with trophoblastic disease. Am. J. Obstet. Gynecol. 1982 [1]). Examples of prior art tests using this marker are, for example, immunological tests described in European patent EP 0 565 541. However, these tests are not reliable since they can give false-negative results which are due, for example, to a low concentration of IGFBP-1 in the amniotic fluid, to a hook effect, to degradations of the protein in the vagina and/or in vitro, or to a matrix effect. They can also generate false-positive results which are due, for example, to the residual presence of IGFBP-1 originating from biological media other than the amniotic fluid, in particular the blood or the decidual cells of the cervix when the latter is mature.

Diagnostic tests based on the detection of alpha-fetoprotein (AFP) using specific antibodies in a vaginal or cervical specimen have also been described. An example of a test of the prior art using this marker is described in the article by Rochelson (Rochelson et al. Rapid assay: possible application in the diagnosis of premature rupture of the membranes. Obstet Gynecol 1983; 62:414-418 [2]).

However, this test is not reliable since it can give false-negative and false-positive results (93% sensitivity and 94% specificity in women at less than 39 weeks of amenorrhea).

Another example of a test of the prior art has been described by Kishida (Kishida et al. Diagnosis of premature rupture of the membranes in preterm patients, using an improved AFP kit: comparison with ROM-Check and/or nitrazine test. European Journal of Obstetrics and Gynecology and Reproductive Biology 69 (1996) 77-82 [12]). Out of a population of patients for whom the term is between 16 and 36 weeks, the sensitivity and the specificity on a vaginal specimen were respectively 96.9% and 92.9%. On a cervical specimen, the sensitivity and the specificity were respectively 100% and 85.7%. Thus, and like other tests of the state of the art, this test is not reliable since it gives false-negative and false-positive results.

Other rupture markers which are less efficient have also been described, such as human chorionic gonadotropin hormone (hCG) (Young-Han Kim et al. Vaginal fluid b-human chorionic gonadotropin level in the diagnosis of premature rupture of membranes. Acta Obstet Gynecol Scand 2005: 84: 802-805 [3]) or prolactin (PRL) (Koninckx PR et al. Prolactin concentration in vaginal fluid: a new method for diagnosing ruptured membranes. Br J Obstet Gynaecol 1981; 88:607-610 [4]).

There is therefore a real need to find a method of detecting rupture of fetal membranes which overcomes these faults, drawbacks and obstacles of the prior art, in particular a method with a better sensitivity and/or a better specificity making it possible to reduce costs and to improve the detection of ruptures of fetal membranes.

### SUMMARY OF THE INVENTION

A feature of embodiments of the present invention is precisely to satisfy these needs and drawbacks of the prior art by providing a method of in vitro detection of rupture of fetal membranes.

In particular, the subject of embodiments of the present invention is a method of in vitro detection of a rupture of fetal membranes comprising a step of simultaneously searching, in a vaginal or cervical specimen, for alpha-fetoprotein (AFP) and for insulin-like growth factor-binding protein 1 (IGFBP-1).

According to embodiments of the invention, the detection of IGFBP-1 can be replaced with the detection of any other protein which has been described as being identical to IGFBP-1, for example placental alpha microglobulin 1 (PAMG-1), placental protein 12, pregnancy associated endometrial alpha1 globulin (alpha1 PEG), and endometrial protein 14 (EP14) (Bell C. Secretory endometrial and decidual proteins: studies and clinical significance of a maternally derived group of pregnancy associated serum proteins. Human Reproduction. Vol. 1-3 129-143. 1986 [13]; Nazimova et al. Blood Serum Content of PAMG-1 Protein Binding Insulin like Growth Factor 1 (Somatomedin C) in Patients with Diabetes Mellitus. Bullet. Experim. Biol. and Med. 166/9 September 1993 [14]).

The inventors have discovered, in particular, that the detection of the two markers is complementary and makes it possible to achieve a higher level of sensitivity. In particular, the inventors have discovered that the simultaneous detection of the two markers represented by alpha-fetoprotein (AFP) and insulin-like growth factor-binding protein 1 (IGFBP-1) makes it possible to increase the specificity and the sensitivity of the detection of the rupture.

According to embodiments of the invention, the term vaginal or cervical specimen is intended to mean, for example, a specimen of vaginal or cervicovaginal secretions which is taken from the wall of the vagina of a pregnant woman, from the posterior fornix of the vagina, or from the cervix.

According to embodiments of the invention, the specimen can be obtained by any method known to those skilled in the art. The specimen can be obtained, for example, by pipetting using a pipette or by rubbing the walls of the vaginal cavity, for example with a sterile swab which has a bud made of cotton or of synthetic fiber. It may, for example, be polyester.

According to embodiments of the invention, the search for AFP and for IGFBP-1 can be carried out, for example, by means of antibodies. The detection of each of these markers can be carried out, for example, by means of monoclonal antibodies, of polyclonal antibodies, and/or of any commercially available antibody capable of binding specifically to AFP or to IGFBP-1.

According to embodiments of the invention, the antibodies directed against AFP (anti-AFP antibodies) can be, for example, anti-AFP antibodies described in international application WO 2004/013180, the antibodies sold by the company Anticorps en Ligne (catalog number ABIN93624), the antibodies sold by the company Arista Biologicals (clone 1, catalog number ABAFP-0401; clone 2, catalog number ABAFP-0402; clone 3, catalog number ABAFP-0403; clone 4, catalog number ABAFP-0404), or the antibodies sold by the company Hytest (clone 5H7, clone 4A3 and clone XA2, catalog number 4F16; polyclonal antibody, catalog number ABAFP-0501).

According to embodiments of the invention, the antibodies directed against AFP may or may not be labeled. The labeling is set out below. The anti-AFP antibodies used in capture and those which are labeled must be directed against different epitopes so that the sandwich system can operate.

According to embodiments of the invention, the concentration of the anti-AFP antibody can be, for example, from 0.1 to 100 μg/ml or from 0.1 to 10 mg/ml.

According to embodiments of the invention, the pH of the solution comprising the anti-AFP antibodies can be from 5 to 10, and more particularly from 6 to 9.

According to embodiments of the invention, the antibodies directed against IGFBP-1 (anti-IGFBP-1 antibodies) can be chosen, for example, from the group comprising the antibodies described in the document Rutanen et al, Biochem Biophys Res Commun 1988; 152: 208 [5] and in Pekonen et al, J immunoassay 1989; 10: 325-337 [6], the anti-IGFBP-1 antibodies sold by the company Lenco technologies (catalog number I-695, I-746 and I-805), the anti-IGFBP-1 antibodies sold by the company Sigma Aldrich (catalog number I 2032, clone 33627.11), the antibodies sold by the company AbD Serotech (catalog number 5345-4859X, clone 6302 (7B11); catalog number 5345-4809X, clone 7B10), the antibodies sold by the company Hytest (catalog number 4I52; clone A8 and clone G2; catalog number 4IG8; clone G5F8 and clone C7B9), the antibodies sold by the company Biospacific (catalog number A61403; catalog number A61400; catalog number A61402), and the antibodies sold by the company Santa Cruz (catalog number A61401136, A61400136 or A61403136).

According to embodiments of the invention, the antibodies directed against IGFBP-1 may or may not be labeled. The labeling is set out below. Advantageously, the anti-IGFBP-1 antibodies used in capture and the anti-IGFBP-1 antibodies that are labeled can be directed against different epitopes.

According to embodiments of the invention, the concentration of the anti-IGFBP-1 antibody can be from 0.1 to 100 μg/ml or from 0.1 to 10 mg/ml.

According to embodiments of the invention, the pH of the solution comprising the anti-IGFPB-1 antibodies can be from 5 to 10, and more particularly from 6 to 9.

According to an embodiment of the invention, the concentration of each antibody and/or the pH of each solution comprising each antibody can make it possible to modulate the threshold for detection of the AFP or the IGFBP-1 protein.

According to an embodiment of the invention, the antibodies directed against AFP or IGFBP-1 can be in dry form.

According to embodiments of the invention, the antibodies directed against AFP or IGFBP-1 can be labeled by any means known to those skilled in the art. This can involve, for example, a chemical molecule, for example a fluorescent label, for example rhodamine or fluorescein isothiocyanate (FITC), an enzyme, for example horseradish peroxidase, alkaline phosphatase, β-galactosidase or glucose 6-phosphate dehydrogenase, which is revealed in the presence of a specific substrate, for example TMB (or 3,3′,5,5′-tetramethylbenzidine), 4-methylumbelliferyl phosphate, colored nanoparticles, for example latex or polystyrene particles, colloidal gold particles, liposomes, carbon or selenium particles, fluorescent nanoparticles containing, for example, lanthanide (europium, for example) chelates, or magnetic nanoparticles or radioactive molecules, for example iodine 125, iodine 133 or tritium.

According to embodiments of the invention, the labeling can be different depending on the specificity of the antibody. For example, the anti-IGFBP-1 antibody can be labeled with a label which is different from that of the anti-AFP antibody. The difference in labeling makes it possible to detect whether AFP alone, IGFBP-1 alone and/or AFP and IGFBP-1 are present in the sample by virtue of the difference in labeling. The different labelings make it possible to distinctly detect AFP and IGFBP-1.

According to embodiments of the invention, the antibodies can be attached beforehand to a solid support. For example, the antibodies directed against AFP or IGFBP-1 can be attached to a membrane, for example a nitrocellulose membrane, the bottom of a multiwell plate and/or any surface known to those skilled in the art which makes it possible to attach at least one antibody.

According to embodiments of the invention, the amount of antibodies directed against AFP or IGFBP-1 that is attached can be adjusted according to the antibody and the protein being sought. For example, when AFP is sought, the amount of antibodies attached can be from 0.1 to 100 μg/ml or from 0.1 to 10 mg/ml; when IGFPB-1 is sought, the amount of antibodies attached can be from 0.1 to 100 μg/ml or from 0.1 to 10 mg/ml.

Control of the amount of antibodies attached makes it possible to adjust the detection threshold for AFP and/or IGFBP-1. Indeed, the greater the amount attached, the lower the detection threshold.

In other words, according to embodiments of the invention, the antibodies can be attached beforehand to a support, for example by deposition or by chemical coupling.

When the antibodies are attached by deposition, they can be deposited, for example, by spraying as a common or distinct surface of anti-AFP antibodies and of anti-IGFBP-1 antibodies as previously defined, and then drying for example, via the method described in Beer et al. Qualification of cellulose nitrate membranes for lateral-flow assays, IVD technology, January 2002. [7]

When the antibodies are attached by chemical coupling, they can be attached, for example, by the method described in Beer et al. Qualification of cellulose nitrate membranes for lateral-flow assays, IVD technology, January 2002. [7]

According to embodiments of the invention, the method of the invention can also comprise a preliminary step of diluting the vaginal or cervical specimen in a diluting solution. The diluting solution can be any solution known to those skilled in the art for diluting these specimens. It can, for example, be a physiological solution, or a buffer solution. It can, for example, also be a solution comprising 50 mM of borate (02102391, Biosolve), 1% BSA (AP-4510-01, Seracare Life Sciences) 0.05% triton X-100 (pH 9.3).

According to embodiments of the invention, the pH of the diluting solution can be from 5 to 10, and more particularly from 6 to 9.

According to embodiments of the invention, the ionic strength of the diluting solution can be from 10 to 1000 mM, and more particularly from 50 to 200 mM.

According to embodiments of the invention, the method can comprise a visualizing step.

According to embodiments of the invention, the visualizing step can be carried out by any method known to those skilled in the art. It can, for example, be a radioimmunological (RIA), immunoradiometric (IRMA), immunoenzymatic (EIA), immunofluorometric (IFMA), time-resolved immunofluorometric, luminoimmunological (LIA) or immunomagnetic method, or a method by immunochromatography or immunofiltration using colored particles (latex, polystyrene, or colloidal). Some of these methods can be carried out as described in Anne Harwood Peruski et al. Immunological Methods for Detection and Identification of Infectious Disease and Biological Warfare Agents. Clinical and diagnostic laboratory immunology, July 2003, p. 506-513. [8]

According to embodiments of the invention, the visualization can, for example, be direct or indirect.

When the visualization is indirect or by competition, use can be made, for example, of a labeled antibody directed against IGFBP-1 or AFP as previously described.

When the visualization is direct (sandwich system), use can be made, for example, of a capture antibody and a labeled second antibody directed against IGFBP-1 and a capture antibody and a labeled second antibody directed against AFP as previously described. The capture antibody and the labeled antibody are directed against different epitopes so as to form a pair that can be used in a sandwich system.

In one particular embodiment, the visualization is direct.

The intensity of the signal of the label depends on the amount and/or on the concentration of AFP and/or of IGFBP-1 present in the specimen.

Depending on the label used, the visualization can be visual or carried out using a device.

When the label is a visible colored label as previously described, the visualization is visual. When the label is, for example, a radioactive label as previously described, the visualization will be carried out, for example, by means of a device for measuring radioactivity, for example a Geiger counter.

The present invention makes it possible to detect AFP in a specimen when its concentration is from 0.5 to 50 ng/ml, more particularly from 1 to 25 ng/ml, and even more particularly from 1 to 2 ng/ml.

The present invention makes it possible to detect AFP in a specimen when its concentration is, for example, greater than or equal to a concentration of between 2.5 and 50 ng/ml, and more particularly between 5 and 25 ng/ml.

In embodiments of the present invention, the detection of AFP can be carried out in a diluted sample as previously defined.

In embodiments of the present invention, the determination of the AFP threshold can be carried out using a preparation of native AFP originating from the company Hytest (Ref. 8F8).

Embodiments of the present invention make it possible to detect IGFBP-1 in a specimen when its concentration is greater than or equal to a concentration of between 5 and 50 ng/ml, and more particularly between 10 and 30 ng/ml.

Embodiments of the present invention also make it possible to detect IGFBP-1 in a specimen when its concentration is greater than or equal to a concentration of between 5 and 30 ng/ml, and more particularly between 5 and 10 ng/ml.

In embodiments of the present invention, the detection of IGFBP-1 can be carried out in a diluted sample as previously described.

In embodiments of the present invention, the determination of the IGFBP-1 threshold can be carried out using a preparation of native IGFBP-1 originating from the company Hytest (Ref. 8IGB1).

According to an embodiment of the invention, the method of can comprise an adjustment of at least one of the parameters chosen from the volume of the diluent buffer medium, the pH of the buffer medium, and the ionic strength of the buffer medium, said adjustment being common to AFP and to IGFBP-1.

According to an embodiment of the invention, the method can comprise an adjustment of at least one of the parameters chosen from the concentration of the capture antibodies and/or the concentration of the labeled antibodies, said adjustment being specific to AFP or to IGFBP-1.

The combination of the adjustment specific to AFP and to IGFBP-1 and/or of the common adjustment makes it possible to obtain complementarity of detection of AFP and of IGFBP-1. The complementarity of the two markers can be, for example, chronological; for example, AFP can be a preferential marker before the 35^{th} week and IGFBP-1 can be a preferential marker after the 37^{th} week. The complementarity can also relate to the performance levels for detection of each of the markers. Thus, according to embodiments of the present invention, the detection thresholds for each of the two markers can be adjusted in such a way that AFP is the most sensitive marker and IGFBP-1 the most specific, and/or vice versa.

According to an embodiment of the present invention, it is also possible to adjust the abovementioned parameters such that the specificity of detection of the markers is identical, i.e. they have the same level of specificity. In this embodiment, the IGFBP-1 marker is statistically the most sensitive, in particular after the 37^{th} week of amenorrhea.

According to embodiments of the invention, the method makes it possible, by virtue of the complementarity of the two markers, to detect a rupture of fetal membranes even if IGFBP-1 is absent, for example if the amniotic fluid has a very low concentration of IGFBP-1 or if the IGFBP-1 has been degraded in vivo or in vitro as has in particular been described in Rutanen et al. Measurement of Insulin-like growth factor binding protein-1 in cervical/vaginal secretions: comparison with the ROM-Check Membrane Immunoassay in the diagnosis of ruptured fetal membranes. Clinica Chimica Acta, 214 (1993), 73-81 [9], by detecting AFP.

According to embodiments of the invention, the method makes it possible, by virtue of the complementarity of the two markers, to steer the diagnosis toward an absence of rupture in the case of an IGFBP-1-positive result that was not due to leaking of amniotic fluid, but to the presence of IGFBP-1 originating from decidual cells of the cervix. This is because, unlike IGFBP-1, AFP is a molecule which is very predominantly of fetal origin and, consequently, does not exhibit this risk of interference. The presence of IGFBP-1 originating from the decidual cells of the cervix limits the value of IGFBP-1; this has been described in particular in the articles: Rutanen et al. Decidual transformation of human extrauterine mesenchymal cells is associated with the appearance of insulin like growth factor binding protein 1. J. Clin. Endocrinol. Metab. 1992; 72: 27-31 [10], Rutanen et al. Evaluation of a rapid strip test for insulin-like growth factor binding protein 1 in the diagnosis of foetal ruptured membranes. Clinica Chimica Acta. 253; (1996) 91-101 [11]. The embodiments of the present invention therefore make it possible to reduce the risk of detection of false positives.

According to an embodiment of the invention, the method can also comprise an additional step of detecting IGFBP-1 alone.

The detection of IGFPB-1 alone in the additional step can be carried out with a higher sensitivity than the detection of IGFBP-1 when carried out simultaneously with that of AFP.

In this embodiment, the detection threshold is such that it makes it possible, in the context of this additional step, to detect IGFBP-1 when its concentration is from 1 to 10 ng/ml, and more particularly from 2 to 5 ng/ml.

In addition in this embodiment, the detection of IGFBP-1 can be carried out in a diluted solution, for example a diluted solution as previously defined.

In this embodiment, the means for detecting IGFBP-1 are those previously defined; they may, for example, be the anti-IGFBP-1 antibodies previously defined.

According to embodiments of the invention, the result is negative, i.e. there is no rupture, when neither IGFBP-1 nor AFP are detected, and positive, i.e. there is rupture, when IGFBP-1 is detected alone or in combination with AFP. When only AFP is detected, the method according to an embodiment of the invention can comprise an additional step of detecting IGFBP-1 alone as previously indicated.

According to this embodiment, the method of the invention makes it possible to eliminate all the false-positive results. In addition, the method of the invention in this embodiment makes it possible to obtain a specificity of 100% and a sensitivity of greater than 84%.

According to embodiments of the invention, the sample can be a sample taken at any time in the pregnancy. For example, the sample can be taken between the 11^{th} week and the end of the pregnancy, for example from 11 to 42 weeks, from 15 to 41, from 20 to 40, from 25 to 40, from 30 to 40, from 35 to 40, or from 37 to 40.

It is possible to obtain a result with certainty, i.e. with a sensitivity and a specificity of 100% on an overall population, with the exception of patients whose term is greater than 37 to 40 weeks, for example 39 weeks with AFP negative and IGFBP-1 negative, and patients whose term is less than or equal to 37 to 40 weeks, for example 39 weeks with AFP positive and IGFBP-1 negative.

Thus, the result obtained makes it possible to conclude that there is no rupture of fetal membranes, without any additional clinical tests. In this embodiment, the method of the invention makes it possible to obtain 100% sensitivity and specificity of the detection method.

The method of embodiments of the invention therefore makes it possible to obtain a reliable result which does not require any additional step of confirmation and which makes it possible to conclude with certainty as to whether or not there is rupture of fetal membranes.

The method of embodiments of the invention therefore makes it possible, on a clearly defined population of patients, i.e. whose term is greater than or less than 39 weeks of amenorrhea, to obtain a reliable result which does not require any additional step of confirmation and which makes it possible to conclude with certainty as to whether or not there is rupture of fetal membranes.

A subject of embodiments of the present invention is also a device, for example an immunochromatographic device, for implementing the method according to the invention, comprising:
a zone (1) for depositing a sample,
a zone (2) comprising labeled anti-IGFBP-1 antibodies and labeled anti-AFP antibodies,
a visualizing zone (3) comprising anti-AFP capture antibodies and anti-IGFBP-1 capture antibodies.

According to embodiments of the invention, the depositing zone (1) can be a zone suitable for the application or the reception of the specimen. This zone can be of any form known to those skilled in the art, for example a reservoir, a cupule, a well, a wick or a flat surface.

According to embodiments of the invention, the depositing zone (1) can be mobile and/or linked to the zone (2) or (3). When the zone (1) is mobile, it can be, for example, used to take the specimen and applied to the zone (2). When the zone (1) is linked to the zone (2) or (3), it can be immersed directly in a container comprising the specimen and/or the specimen can be applied to this zone. An example of this device is illustrated below.

According to an embodiment of the invention, the zones (1) and (2) are located on the same support.

According to embodiments of the invention, the materials of the zones (1) to (3) can be identical or different. The materials can be any material known to those skilled in the art, for example a material chosen from the group comprising absorbent paper, cotton, cellulose, glass fiber, and a nitrocellulose membrane.

In a particular embodiment, the depositing zone (1) can be an absorbent paper or glass fiber.

In a particular embodiment, the zone (2) comprising labeled anti-IGFBP-1 antibodies and labeled anti-AFP antibodies can be glass fiber.

In a particular embodiment, the visualizing zone (3) can be a nitrocellulose membrane.

According to an embodiment of the invention, the anti-AFP or anti-IGFBP-1 capture antibodies can be, for example, attached directly to the support of the zone (3), for example by chemical coupling and/or by deposition as previously described.

According to embodiments of the invention, the zone of attachment of the AFP capture antibody and of the IGFBP-1 capture antibody can be identical or different. In a particular embodiment, the zone of attachment of the AFP capture antibody is different than that of the IGFBP-1 capture antibody. They can, for example, be two parallel bands each comprising a different capture antibody.

According to embodiments of the invention, the abovementioned various zones (1) to (3) can be attached to a solid support, for example to laminated cards, and/or included in a container comprising an orifice at the level of the zone (3) allowing the visualization of the result and an orifice at the level of the zone (1) for depositing the sample, allowing the deposition of the sample. An example of this device is illustrated below.

According to an embodiment of the invention, the device can also comprise an absorption zone (4). According to an embodiment of the invention, the absorption zone (4) can be any absorbent solid support known to those skilled in the art. It can, for example, be an absorbable blotting paper, a sponge, cotton wool, lightweight felt or a synthetic textile.

According to an embodiment of the invention, the supports of the zones (1) and (2) overlap at one of their ends, and the other end of the zone (2) overlaps with one end of the zone (3). The overlapping of the various zones (1) to (3) makes it possible, when the specimen is applied and/or when the free end of the zone (1) is immersed in the specimen, for the specimen to migrate in the various zones via capillary action. In a particular embodiment, the zones (1) and (2) are arranged on the same support. An example of this device is illustrated below.

In a particular embodiment, the absorption zone (4) overlaps with the free end of the zone (3). Thus, the zone (4) allows accelerated migration of the specimen through the various zones of the device. The zone (4) makes it possible to absorb the excess liquid of the specimen.

According to embodiments of the invention, the various zones (1) to (4) can be independently covered with a protective film. This can, for example, be a plastic film, for example a polyvinyl chloride (PVC) film, or a biodegradable film, for example a polycaprolactone (PCL), polyvinyl alcohol (PVA) or polylactic acid (PLA) film.

The film makes it possible to independently protect the various zones of the device of the invention.

According to embodiments of the invention, the film can be a single film or several films independently covering one of said zones (1) to (4).

According to embodiments of the invention, the film or films can partially cover the device of the invention, thus leaving certain zones uncovered. According to embodiments of the invention, the film or films can be a detachable film or films which can be removed before the use of the device. According to an embodiment of the invention, the film can comprise an inscription at its surface which makes it possible to identify the device.

A subject of embodiments of the present invention is also a kit for implementing the method of the invention, comprising:

anti-AFP and anti-IGFBP-1 capture antibodies as previously described,

labeled anti-AFP and anti-IGFBP-1 antibodies as previously defined.

According to embodiments of the invention, the kit may also comprise a sampling means, for example a swab, as previously defined.

According to embodiments of the invention, the kit may also comprise a solid support on which capture antibodies as previously defined can be attached.

A subject of embodiments of the present invention is also the use of the device according to the present invention for detecting rupture of fetal membranes as previously defined.

Embodiments of the present invention make it possible to detect ruptures of fetal membranes whatever their size, for example whether they are microruptures and/or macroruptures, and/or whatever the volume of amniotic fluid sampled.

Embodiments of the present invention make it possible to reduce the probability of obtaining false-negative results and also false-positive results.

In addition, embodiments of the present invention make it possible to detect ruptures of fetal membranes whatever the stage of the pregnancy.

Embodiments of the present invention also allows reliable detection whatever the stage of the pregnancy, the variation in concentration of AFP and/or of IGFBP-1 in the amniotic fluid and/or the volume of amniotic fluid present in the vaginal or cervical specimen.

Other features and advantages may further become apparent to those skilled in the art on reading the examples below, illustrated by the appended figures, given by way of illustration.

### BRIEF DESCRIPTION OF THE DRAWINGS

FIG. 1 represents a front view (A) and a side view (B) of a device according to an embodiment of the invention.

FIG. 2 represents a device included in a container according to an embodiment of the invention.

### DETAILED DESCRIPTION

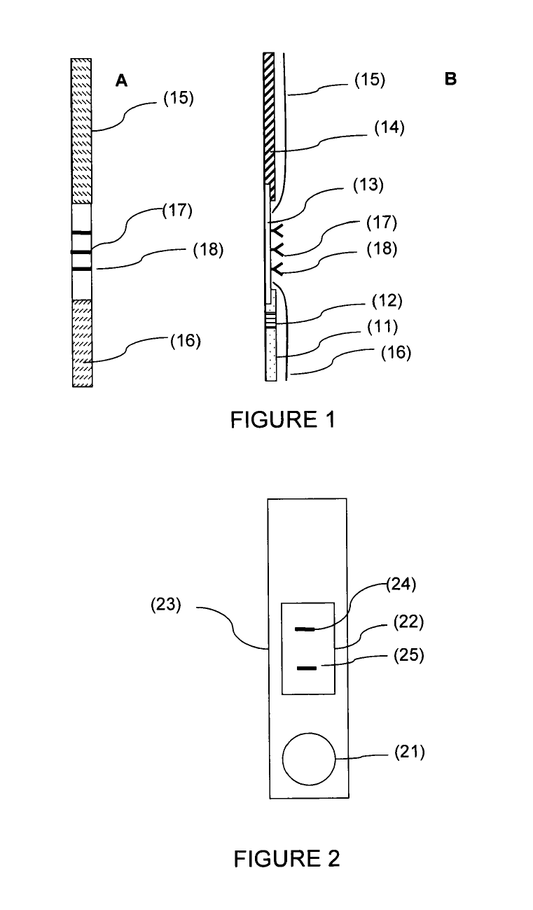

Referring to FIG. 1, and as discussed above in the Summary section, according to embodiments of the device, for example an immunochromatographic device, for implementing the method according to the invention, includes a zone (1) for depositing a sample, a zone (2) comprising labeled anti-IGFBP-1 antibodies and labeled anti-AFP antibodies, and a visualizing zone (3) comprising anti-AFP capture antibodies and anti-IGFBP-1 capture antibodies.

The depositing zone (1) can be a zone suitable for the application or the reception of the specimen. This zone can be of any form known to those skilled in the art, for example a reservoir, a cupule, a well, a wick or a flat surface. According to embodiments of the invention, the depositing zone (1) can be mobile and/or linked to the zone (2) or (3). When the zone (1) is mobile, it can be, for example, used to take the specimen and applied to the zone (2). When the zone (1) is linked to the zone (2) or (3), it can be immersed directly in a container comprising the specimen and/or the specimen can be applied to this zone.

According to an embodiment of the invention, the supports of the zones (1) and (2) overlap at one of their ends, and the other end of the zone (2) overlaps with one end of the zone (3). The overlapping of the various zones (1) to (3) makes it possible, when the specimen is applied and/or when the free end of the zone (1) is immersed in the specimen, for the specimen to migrate in the various zones via capillary action. In a particular embodiment, the zones (1) and (2) are arranged on the same support.

The materials of the zones (1) to (3) can be identical or different. The materials can be any material known to those skilled in the art, for example a material chosen from the group comprising absorbent paper, cotton, cellulose, glass fiber, and a nitrocellulose membrane.

According to an embodiment of the invention, the device can also comprise an absorption zone (4). According to an embodiment of the invention, the absorption zone (4) can be any absorbent solid support known to those skilled in the art. It can, for example, be an absorbable blotting paper, a sponge, cotton wool, lightweight felt or a synthetic textile. In a particular embodiment, the absorption zone (4) overlaps with the free end of the zone (3). Thus, the zone (4) allows accelerated migration of the specimen through the various zones of the device. The zone (4) makes it possible to absorb the excess liquid of the specimen.

According to embodiments of the invention, the various zones (1) to (4) can be independently covered with a protective film (15,16). This can, for example, be a plastic film, for example a polyvinyl chloride (PVC) film, or a biodegradable film, for example a polycaprolactone (PCL), polyvinyl alcohol (PVA) or polylactic acid (PLA) film.

In a particular non-limiting embodiment shown in FIG. 1, the device can comprise zone (1) made of glass fiber (11), zone (2) made of glass fiber (12) comprising labeled anti-IGFBP-1 antibodies and labeled anti-AFP antibodies (12), zone (3) made of a nitrocellulose membrane (13) comprising anti-AFP capture antibodies (17) and anti-IGFBP-1 capture antibodies (18) and zone (4) made of absorbent paper (14), and a film (15) (16).

According to embodiments of the invention, the abovementioned various zones (1) to (3) can be attached to a solid support, for example to laminated cards, and/or included in a container comprising an orifice at the level of the zone (3) allowing the visualization of the result and an orifice at the level of the zone (1) for depositing the sample, allowing the deposition of the sample. An example of this device is illustrated in FIG. 2. Referring to FIG. 2, a device included in a container (23), according to an embodiment of the invention, can comprise an orifice (21) at the level of the zone (1) and an orifice (22) at the level of the zone (3) comprising anti-AFP capture antibodies (24) and anti-IGFBP-1 capture antibodies (25).

### EXAMPLES

### Example 1

### Method of Detecting Rupture of Fetal Membranes by Means of an Immunofiltration Device

Anti-AFP antibodies (ABAFP-0404, clone 4, Arista Biologicals) diluted to 0.75 mg/ml in a PBS buffer and also anti-IGFBP-1 antibodies (I 2032, clone 33627.11, Sigma Aldrich) diluted to 0.75 mg/ml in a PBS buffer are prepared.

The antibodies are deposited, using a reagent-distributing automated device (Isoflow Dispenser, Imagene Technology Inc. (registered trademark)), in the form of parallel lines on a nitrocellulose membrane, and then dried in an incubator at 37° C. under an atmosphere with a controlled degree of humidity of less than 30%. The membrane is then cut up into multiple strips approximately 1 centimeter wide and 2.5 centimeters long. Each strip is then placed on an absorbent support placed in a plastic base. A plastic cover which fits the base is then placed over the base and closed by manual pressure. The plastic cover has a window opening, the surface area of which is less than the surface area of the membrane. The membrane was positioned such that the open zone of the window of the cover is entirely located above the membrane. The cover is designed in such a way that it exerts a pressure on the membrane and the absorbent support so that the various elements of the device keep the position defined during the closing of the cover.

A specimen is taken from a pregnant woman at 24 weeks of amenorrhea, using a sterile swab which has a polyester bud.

The bud is then placed in a 5 ml screw-cap tube (Dustcher) comprising 2 ml of an extraction solution containing 50 mM of borate (02102391, Biosolve), 1% bovine serum albumin (BSA) (AP-4510-01, Seracare Life Sciences) and 0.05% triton X-100 (pH 9.3), and stirred by circular movements for 30 seconds to one minute. The swab is then removed from the tube.

The extraction solution is then poured into the window of the cover. The solution is filtered through the membrane and absorbed by the absorbent support. The AFP and/or the IGFBP-1 contained in the extraction solution bind(s) with the respective capture antibodies. During the next step, a visualizing reagent containing anti-AFP antibodies, clone 3, Arista Biologicals, ABAFP-0403, and anti-IGFBP-1 antibodies, clone 6302 (7B11), AbD Serotech, 5345-4859X, which have paratopes that are different than the capture antibodies and are labeled with colloidal gold, bind to the captured AFP and/or IGFBP-1 antigens. This leads to the appearance of a mauve coloration on the test lines. A step of washing with a washing solution containing, for example, PBS and 10% Tween-20 is carried out by pouring washing solution into the window in order to remove any nonspecific binding of the colloidal gold particles. The presence of mauve-colored lines on the AFP and/or IGFBP-1 lines indicates rupture of fetal membranes.

### Example 2

### Immunochromatographic Device for Detecting Rupture of Fetal Membranes

The strips are prepared from laminated cards which are 30 cm×9.5 cm (CNPC-SS12 R-032/2, MDI (registered trademark)) consisting of a plastic support covered with an adhesive layer on which the nitrocellulose membrane, the absorbent paper and the glass fiber are assembled.

Using a reagent-distributing automated device (Isoflow Dispenser, Imagene Technology Inc. (registered trademark)), goat anti-mouse IgG antibodies (ABGAM-0500, Arista Biologicals (registered trademark)) diluted to 4 mg/ml in a PBS buffer, anti-AFP antibodies (ABAFP-0404, clone 4, Arista Biologicals) diluted to 0.75 mg/ml in a PBS buffer and anti-IGFBP-1 antibodies (I 2032, clone 33627.11, Sigma Aldrich (registered trademark)) diluted to 0.75 mg/ml in a PBS buffer are deposited with a flow rate of 0.5 μl/cm in the form of parallel lines 5 mm apart and each from 1 to 2 mm wide, on a nitrocellulose membrane which is 25 mm wide (zone (1)).

After these searching means have been deposited, these cards are then placed in an incubator at 37° C. for 30 minutes under an atmosphere with a controlled degree of humidity of less than 30% in order to dry them.

The conjugate mixture, i.e. a solution comprising labeled antibodies, is prepared from the anti-IGFBP-1 (5345-4859X, clone 6302 (7B11), AbD Serotech (registered trademark)) and anti-AFP (ABAFP-0403, clone 3, Arista Biologicals (registered trademark)) antibodies coupled beforehand to latex particles (DBK1040CB, diameter 0.39 μm, Duke Scientific Corporation (registered trademark)) according to the indications of the supplier or the following instructions:

The latex particles (150 μl) are washed twice with 1 ml of PBS by centrifugation for 30 minutes at 14 000 revolutions per minute (rpm) in 1.5 ml eppendorf (registered trademark) tubes. At the final washing step, the latex particles are resuspended in the same solution. The carboxylated parts of the beads are then activated with 50 μg of N-(3-dimethylaminopropyl)-N-ethylcarbodiimide hydrochloride (EDC, E1769, Sigma-Aldrich (registered trademark)). After 15 minutes of activation, the antibody solution is added in a proportion of 10 μg/mg of latex particles. The resulting solution is carefully stirred for 2 h at ambient temperature on a rotary mixer suitable for 1.5 ml tubes. A step of centrifugation for 15 minutes at 14 000 revolutions per minute subsequently makes it possible to recover the latex particles conjugated to the antibodies. In order to avoid nonspecific interactions, the pellet is taken up in 1 ml of a saturation solution (pH 7.4) containing 50 mM Na₂HPO₄ (S7907, Sigma Aldrich (registered trademark)), 1% BSA (AP-4510-01, Seracare Life Sciences (registered trademark)). The solution is stirred on the rotary mixer for 1 hour at ambient temperature and then placed in an incubator for 30 minutes at 37° C. The solution is centrifuged one last time for 15 minutes at 14 000 revolutions per minute. The resulting pellet is resuspended in the same saturation solution.

Added to 250 μl of solution of IGFBP-1 conjugate (labeled anti-IGFBP-1 antibodies) supplemented with 250 μl of AFP conjugate (labeled anti-AFP antibodies) is one volume of the saturation solution (pH 7.4) containing 50 mM Na₂HPO₄ (S7907, Sigma Aldrich (registered trademark)), 1% bovine serum albumin (BSA) (AP-4510-01, Seracare Life Sciences) qs for 1 ml, 20% of sucrose (S8501, Sigma Aldrich) and 5% of trehalose (T9531, Sigma Aldrich (registered trademark)). The resulting mixture is stirred for 15 minutes at ambient temperature on the rotary mixer suitable for 1.5 ml tubes.

The antibodies were thus coupled with said latex particles and correspond to examples of labeled antibodies.

The mixture of antibodies coupled to the latex microparticles is subsequently sprayed with a flow rate of 4 μl/cm onto sheets of glass fiber (zone (3)) having dimensions of 30 cm×3.2 cm (#8964, Ahlstrom, Arista Biologicals (registered trademark)), pretreated with a 0.1 M solution of B₄Na₂O₇ (B3545, Sigma Aldrich (registered trademark)), 1% triton X-100 (X-100, Sigma Aldrich (registered trademark)), pH 8.4.

The attaching of this mixture of conjugates to the sheet of glass fiber is carried out with the same Isoflow Dispenser (registered trademark) apparatus in accordance with the indications of the supplier. After spraying, the glass fiber sheets are dried for half an hour at 37° C.

The cards are assembled by adhesive bonding of the conjugate-impregnated glass fiber onto the lower adhesive part of the card with the glass fiber sheet overlapping the nitrocellulose membrane by 1 to 3 mm. In the same way, a high absorbency paper (zone 2) (absorbent pad, Ahlstrom 222, MAPDS-0100, Arista Biologicals) with dimensions of 30 cm×4.2 cm is placed on the upper part of the card, overlapping the nitrocellulose membrane by 1 to 3 mm in order to be able to create a migration flow. A self-adhesive label is then placed on the two types of paper and has the function of protecting and identifying the strips.

The card thus assembled is cut into small strips 4 mm wide using a guillotine (GCI-800, Zeta Corporation) or a rotary multiblade device (rotary cutter, Arista Biologicals Inc). A strip is represented by the scheme below:

### Example 3

### Example of Use of a Device for Implementing the Method of the Invention

1. A solution of 2.5 ml of 50 mM borate (02102391, Biosolve), 1% BSA (AP-4510-01, Seracare Life Sciences) and 0.05% triton X-100 (pH 9.3) was introduced into a 5 ml screw-cap tube (015610, Dustcher),
2. the sample was taken with a sterile swab having a polyester bud and deposited on the device described in example 2,
3. the device obtained was introduced into the abovementioned screw-cap tube,
4. the result is obtained after 10 minutes of migration.
   The test is positive when at least one of the two bands corresponding to AFP and to IGBPF-1 is colored.

When the test is positive, the veracity of the test can be verified by a complete gynecological examination making it possible to confirm or refute the result.

### Example 4

### Comparison of the Protease-Stability of AFP and of IGFBP-1 Carried Out Using a Device Implementing the Method of the Invention

In this example, the immunochromatographic device described in example 2 is modified and uses a system of labeling with colloidal gold for detecting the IGFBP-1 and AFP molecules. The pairs of anti-IGFBP-1 and anti-AFP antibodies were selected from the list of antibodies presented in the description of the invention.

Two microliters and ten microliters of an amniotic fluid are deposited in two distinct tubes each containing 2.5 milliliters of extraction solution. 200 microliters of fresh urine are then added to each of the two tubes. The strips are then deposited in the extraction solutions according to the modes described in example 3 above. The tubes containing the extraction solution are then placed at 37° C. and are tested at 24 hours, 48 hours and 6 days. The amniotic fluid sample was selected by virtue of its property of generating starting signals (at time T0) of identical intensity on the AFP and IGFBP-1 bands.

Regarding the tube containing two microliters of amniotic fluid, after 24 hours of incubation, the IGFBP-1 signal decreased significantly, whereas the AFP signal decreased only very slightly. After 48 hours of incubation, the IGFBP-1 signal disappeared, whereas the intensity of the AFP signal did not change compared with the previous reading at 24 hours. At 6 days, the AFP signal remains visible and unchanged, whereas the IGFBP-1 signal remains absent.

Regarding the tube containing ten microliters of amniotic fluid, after 24 hours of incubation, the IGFBP-1 and AFP signals remain unchanged. After 48 hours of incubation, the IGFBP-1 decreased, whereas the AFP signal did not change. At 6 days, the AFP signal remains unchanged, whereas the IGFBP-1 signal was very greatly reduced and visible only in trace form.

### Example 5

### Comparison of the Performance Levels of the AFP/IGFBP-1 Combination vs IGFBP-1 Alone

A prospective study was carried out from Nov. 17, 2010 to Jan. 15, 2011 among a group of 102 patients. All the patients who consulted for suspected flow of amniotic fluid after 12 weeks of amenorrhea (WA) and for whom the clinician, after clinical examination with a speculum, found an indication to carry out a biological test to search for amniotic fluid, were included. Patients exhibiting metrorrhagia were not eligible. The taking of the specimen consisted in collecting the secretions present in the vaginal fornix with two swabs in parallel: the sterile polyester swab (25-806, Pur-Wraps (registered trademark) Puritan) used with the device implementing the method of the invention and the ACTIM® (registered trademark) PROM swab (25-806, Pur-Wraps (registered trademark) Puritan) with the ACTIM PROM test.

ACTIM® (registered trademark) PROM (Medix Biochemica (registered trademark)) is an immunochromatographic test which uses two antibodies directed against human IGFBP-1. One is attached to blue latex particles and the other is immobilized on the membrane of the strip forming a capture line. The swab is immersed in 0.5 ml of the extraction solution provided in the kit (phosphate buffered solution containing bovine serum albumin, protease inhibitors and preservatives). It is stirred vigorously for 10 seconds. The strip is immersed until the liquid front reaches the level of the results zone, and it is then withdrawn. The reading is carried out after 5 minutes of migration.

A result is considered to be positive if two blue lines appear, thereby reflecting the presence of IGFBP-1 at a level greater than 25 μg/ml.

The device implementing the method of the invention is an immunochromatographic test which uses a system of labeling anti-AFP and anti-IGFBP-1 antibodies with colloidal gold and of immobilizing AFP and IGFBP-1 antibodies on the membrane of the strip for detecting IGFBP-1 and AFP molecules on two distinct lines. In this example, the swab was immersed for 10 seconds in a 2 ml screw-cap tube (015610, Dustcher) with 0.5 ml of a 50 mM solution of Na₂HPO₄ (P4417, Sigma Aldrich) containing 1% BSA (AP-4510-01, Seracare Life Sciences) and 0.05% triton (X-100, Sigma Aldrich), pH 7.4. This diluted specimen was stored at −20° C. until all the samples had been selected. When the test was carried out, the device implementing the method of the invention was introduced into the abovementioned screw-cap tube. The result was read after 10 minutes of migration.

The strips were prepared from laminated cards with dimensions of 30 cm×9.5 cm (CNPC-SS12 R-032/2, MDI (registered trademark)) consisting of a plastic support covered with an adhesive layer on which the nitrocellulose membrane, the absorbent paper and the glass fiber were assembled.

Using a reagent-distributing automated device (Isoflow Dispenser, Imagene Technology Inc. (registered trademark)), goat anti-mouse IgG antibodies (ABGAM-0500, Arista Biologicals (registered trademark)) diluted to 2 mg/ml in a PBS buffer, pH 7.4, containing 0.015M Na₂HPO₄ (P4417, Sigma (registered trademark)) and 0.15M NaCl (S7653, Sigma (registered trademark)), anti-AFP antibodies which were chosen from the following list: the company Anticorps en Ligne (catalog number ABIN93624), the antibodies sold by the company Arista Biologicals (clone 1, catalog number ABAFP-0401; clone 2, catalog number ABAFP-0402; clone 3, catalog number ABAFP-0403; clone 4, catalog number ABAFP-0404), the antibodies sold by the company Hytest (clone 5H7, clone 4A3 and clone XA2, catalog number 4F16; polyclonal antibody, catalog number ABAFP-0501) diluted to 1 mg/ml in a PBS buffer, and also anti-IGFBP-1 antibodies which were chosen from the following list: the anti-IGFBP-1 antibodies sold by the company Lenco technologies (catalog number I-695, I-746 and I-805), the anti-IGFBP-1 antibodies sold by the company Sigma Aldrich (catalog number I 2032, clone 33627.11), the antibodies sold by the company AbD Serotech (catalog number 5345-4859X, clone 6302 (7B11); catalog number 5345-4809X, clone 7B10) and the antibodies sold by the company Hytest (catalog number 4I52; clone A8 and clone G2; catalog number 4IG8; clone G5F8 and clone C7B9), diluted to 0.5 mg/ml in a PBS buffer, were deposited with a flow rate of 1 μl/cm in the form of parallel lines 5 mm apart and each from 1 to 2 mm wide, on a nitrocellulose membrane 25 mm wide (zone (1)).

After deposition of these searching means, these cards were then placed in an incubator at 37° C. for 30 minutes under an atmosphere with a controlled degree of humidity of less than 30% in order to dry them.

The conjugate mixture, i.e. a solution comprising labeled antibodies, was prepared from the anti-IGFBP-1 antibodies which were chosen from the following list: the anti-IGFBP-1 antibodies sold by the company Lenco technologies (catalog number I-695, I-746 and I-805), the anti-IGFBP-1 antibodies sold by the company Sigma Aldrich (catalog number I 2032, clone 33627.11), the antibodies sold by the company AbD Serotech (catalog number 5345-4859X, clone 6302 (7B11); catalog number 5345-4809X, clone 7B10) and the antibodies sold by the company Hytest (catalog number 4I52; clone A8 and clone G2; catalog number 4IG8; clone G5F8 and clone C7B9), and the anti-AFP antibodies which were chosen from the following list: the company Anticorps en Ligne (catalog number ABIN93624), the antibodies sold by the company Arista Biologicals (clone 1, catalog number ABAFP-0401; clone 2, catalog number ABAFP-0402; clone 3, catalog number ABAFP-0403; clone 4, catalog number ABAFP-0404) and the antibodies sold by the company Hytest (clone 5H7, clone 4A3 and clone XA2, catalog number 4F16; polyclonal antibody, catalog number ABAFP-0501), coupled beforehand to colloidal gold particles according to the following protocol:

1.0 ml of a 1% gold chloride solution (G4022, Sigma Aldrich) was added to 100 ml of distilled water. The water was heated to boiling point and 2.5 ml of 1% sodium citrate solution (S1804, Sigma Aldrich) were added. The solution became colorless and then turned purple. After one minute, the solution was heated until it became cherry red. 9.5 ml of 1% sodium citrate (S1804, Sigma Aldrich) were added. The water was heated to boiling point and 8.6 ml of 1% gold chloride solution (G4022, Sigma Aldrich) were added. The solution became dark blue, virtually black, then purple, and was then left to cool to ambient temperature.

Firstly, the pH of 10 ml of the purple colloidal gold solution previously obtained was adjusted to pH 7.8 using a 0.2M potassium carbonate solution (269619, Sigma Aldrich) and, secondly, the pH of 10 ml of purple colloidal gold solution was adjusted to pH 7.3 using a 0.2M potassium carbonate solution (269619, Sigma Aldrich). 200 μg of anti-IGFBP-1 were added to 1 ml of distilled water and then 10 ml of purple colloidal gold solution at the pH adjusted to 7.8 were rapidly added. 200 μg of anti-AFP were added to 1 ml of distilled water and then 10 ml of purple colloidal gold solution at the pH adjusted to 7.3 were rapidly added. These tubes were placed on a rotary mixer (SB2, Stuart (registered trademark)) for 20 minutes. 1 ml of 10% BSA solution (AP-4510-01, Seracare Life Sciences (registered trademark)) and 50 μl of 10% PEG solution (81275, Sigma Aldrich (registered trademark)) were added. The 2 tubes were centrifuged (5804, Eppendorf (registered trademark)) at 4000 rcf for 30 minutes. The supernatant was drawn off using a vacuum pump (159600, Brand (registered trademark)) and the pellet was taken up with 1 ml of resuspension buffer (pH 8.0) containing 20 mM Tris base (26-128-3094, Euromedex (registered trademark)), 50 mM NaCl (S7653, Sigma Aldrich (registered trademark)), 0.2% BSA (AP-4510-01, Seracare Life Sciences (registered trademark)), 10% sucrose (S8501, Sigma Aldrich) and 5% trehalose (T9531, Sigma Aldrich (registered trademark)).

500 μl of the saturation solution (pH 7.4) containing 50 mM Na₂HPO₄ (S7907, Sigma Aldrich (registered trademark)), 1% of bovine serum albumin (BSA) (AP-4510-01, Seracare Life Sciences), 20% of sucrose (S8501, Sigma Aldrich) and 5% of trehalose (T9531, Sigma Aldrich (registered trademark)) were added to 250 μl of solution of IGFBP-1 conjugate supplemented with 250 μl of AFP conjugate. The resulting mixture was stirred for 15 minutes at ambient temperature on the rotary mixer suitable for 1.5 ml tubes.

The antibodies were thus coupled with said colloidal gold particles and correspond to examples of labeled antibodies.

The mixture of antibodies coupled to the gold microparticles was then sprayed with a flow rate of 4 μl/cm onto sheets of glass fiber (zone (3)) with dimensions of 30 cm×3.2 cm (#8964, Ahlstrom, Arista Biologicals (registered trademark)), pretreated with a 0.1 M solution of B₄Na₂O₇ (B3545, Sigma Aldrich (registered trademark)), 1% triton X-100 (X-100, Sigma Aldrich (registered trademark)), pH 8.4.

The attaching of this mixture of conjugates to the glass fiber sheet was carried out with the same Isoflow Dispenser (registered trademark) apparatus in accordance with the indications of the supplier. After spraying, the glass fiber sheets were dried for an hour and a half at 37° C.

The cards were assembled by adhesive bonding of the conjugate-impregnated glass fiber onto the lower adhesive part of the card with the glass fiber sheet overlapping the nitrocellulose membrane by 1 to 3 mm. In the same way, a high absorbency paper (zone 2) (absorbent pad, Ahlstrom 222, MAPDS-0100, Arista Biologicals) with dimensions of 30 cm×4.2 cm was placed on the upper part of the card, overlapping the nitrocellulose membrane by 1 to 3 mm in order to create a migration flow. A self-adhesive label was then placed on the two types of paper and has the function of protecting and identifying the strips.

The card thus assembled was cut into strips 4 mm wide using a guillotine (GCI-800, Zeta Corporation) or a rotary multiblade device (rotary cutter, Arista Biologicals Inc).

Table 1 below gives details of the examples of the results obtained and also the corresponding interpretations by the obstetricians:

| | | Method of the | | | | |
|---|---|---|---|---|---|---|
| Sample | | state of the art | Device of the invention | | | Obstetrician's |
| No. | TERM | INTERPRETATION | IGFBP-1 | AFP | INTERPRETATION | opinion |
| 101 | 27 WA | NEGATIVE | NEGATIVE | NEGATIVE | NEGATIVE | NEGATIVE |
| 103 | 35 WA | POSITIVE | POSITIVE | POSITIVE | POSITIVE | POSITIVE |
| 104 | NC | NEGATIVE | NEGATIVE | NEGATIVE | NEGATIVE | NEGATIVE |
| 105 | 28 WA | NEGATIVE | NEGATIVE | NEGATIVE | NEGATIVE | NEGATIVE |
| 106 | 26 WA | NEGATIVE | NEGATIVE | NEGATIVE | NEGATIVE | NEGATIVE |
| 107 | 39 WA | POSITIVE | POSITIVE | POSITIVE | POSITIVE | POSITIVE |
| 109 | 41 WA | NEGATIVE | NEGATIVE | NEGATIVE | NEGATIVE | NEGATIVE |
| 111 | 40 WA | NEGATIVE | NEGATIVE | NEGATIVE | NEGATIVE | NEGATIVE |
| 112 | 40WA + 4 d | POSITIVE | POSITIVE | NEGATIVE | POSITIVE | POSITIVE |
| 113 | 30 WA | NEGATIVE | NEGATIVE | NEGATIVE | NEGATIVE | NEGATIVE |
| 114 | 36 WA | POSITIVE | POSITIVE | POSITIVE | POSITIVE | POSITIVE |
| 115 | 34WA + 4 d | NEGATIVE | POSITIVE | POSITIVE | POSITIVE | POSITIVE |
| 116 | 22 WA | POSITIVE | POSITIVE | POSITIVE | POSITIVE | POSITIVE |
| 117 | 26 WA | POSITIVE | POSITIVE | POSITIVE | POSITIVE | POSITIVE |
| 118 | 40 WA | POSITIVE | POSITIVE | POSITIVE | POSITIVE | POSITIVE |
| 119 | 40 WA + 2 d | POSITIVE | NEGATIVE | NEGATIVE | NEGATIVE | NEGATIVE |
| 120 | 40 WA | POSITIVE | POSITIVE | POSITIVE | POSITIVE | POSITIVE |
| 122 | 36 WA | POSITIVE | POSITIVE | POSITIVE | POSITIVE | POSITIVE |
| 123 | 39 WA | NEGATIVE | NEGATIVE | NEGATIVE | NEGATIVE | NEGATIVE |
| 124 | 37 WA | NEGATIVE | NEGATIVE | NEGATIVE | NEGATIVE | NEGATIVE |
| 125 | 41 WA | NEGATIVE | NEGATIVE | NEGATIVE | NEGATIVE | NEGATIVE |
| 126 | 39WA + 2 d | POSITIVE | POSITIVE | NEGATIVE | POSITIVE | POSITIVE |
| 129 | 30 WA | NEGATIVE | NEGATIVE | NEGATIVE | NEGATIVE | NEGATIVE |
| 130 | 33 WA | NEGATIVE | NEGATIVE | NEGATIVE | NEGATIVE | NEGATIVE |
| 131 | 12 WA | NEGATIVE | NEGATIVE | NEGATIVE | NEGATIVE | NEGATIVE |
| 132 | 37 WA | NEGATIVE | NEGATIVE | NEGATIVE | NEGATIVE | NEGATIVE |
| 133 | 31 WA | NEGATIVE | NEGATIVE | NEGATIVE | NEGATIVE | NEGATIVE |
| 134 | 38 WA | NEGATIVE | NEGATIVE | NEGATIVE | NEGATIVE | NEGATIVE |
| 135 | 33 WA | NEGATIVE | NEGATIVE | NEGATIVE | NEGATIVE | NEGATIVE |
| 136 | 41WA + 4 d | POSITIVE | POSITIVE | NEGATIVE | POSITIVE | POSITIVE |
| 137 | 37WA + 6 d | POSITIVE | POSITIVE | NEGATIVE | POSITIVE | POSITIVE |
| 139 | 30 WA | NEGATIVE | NEGATIVE | NEGATIVE | NEGATIVE | NEGATIVE |
| 140 | 41 WA | NEGATIVE | NEGATIVE | NEGATIVE | NEGATIVE | NEGATIVE |
| 141 | 41 WA | NEGATIVE | NEGATIVE | NEGATIVE | NEGATIVE | POSITIVE |
| 143 | 40 WA | POSITIVE | POSITIVE | POSITIVE | POSITIVE | POSITIVE |
| 144 | 40 WA | NEGATIVE | NEGATIVE | NEGATIVE | NEGATIVE | NEGATIVE |
| 145 | 23 WA | NEGATIVE | NEGATIVE | NEGATIVE | NEGATIVE | NEGATIVE |
| 146 | 30 WA | NEGATIVE | NEGATIVE | NEGATIVE | NEGATIVE | NEGATIVE |
| 147 | 40 WA | POSITIVE | POSITIVE | POSITIVE | POSITIVE | POSITIVE |
| 148 | 41 WA | POSITIVE | POSITIVE | POSITIVE | POSITIVE | POSITIVE |
| 150 | 38 WA | NEGATIVE | NEGATIVE | NEGATIVE | NEGATIVE | NEGATIVE |
| 151 | 16 WA | NEGATIVE | NEGATIVE | NEGATIVE | NEGATIVE | NEGATIVE |
| 152 | 16 WA | NEGATIVE | NEGATIVE | NEGATIVE | NEGATIVE | NEGATIVE |
| 154 | 36 WA | NEGATIVE | NEGATIVE | NEGATIVE | NEGATIVE | NEGATIVE |
| 155 | 40 WA | NEGATIVE | NEGATIVE | NEGATIVE | NEGATIVE | NEGATIVE |
| 156 | 34 WA | NEGATIVE | NEGATIVE | NEGATIVE | NEGATIVE | NEGATIVE |
| 157 | 38 WA | NEGATIVE | NEGATIVE | NEGATIVE | NEGATIVE | NEGATIVE |
| 158 | 36 WA | NEGATIVE | NEGATIVE | NEGATIVE | NEGATIVE | NEGATIVE |
| 159 | 29 WA | NEGATIVE | NEGATIVE | NEGATIVE | NEGATIVE | NEGATIVE |
| 161 | 18 WA | NEGATIVE | NEGATIVE | NEGATIVE | NEGATIVE | NEGATIVE |
| 163 | 32 WA | POSITIVE | NEGATIVE | NEGATIVE | NEGATIVE | NEGATIVE |
| 164 | 39WA + 6 d | POSITIVE | NEGATIVE | NEGATIVE | NEGATIVE | POSITIVE |
| 165 | 40 WA | POSITIVE | NEGATIVE | NEGATIVE | NEGATIVE | POSITIVE |
| 166 | 41 WA + 3 d | NEGATIVE | NEGATIVE | NEGATIVE | NEGATIVE | NEGATIVE |
| 167 | 32 WA | NEGATIVE | NEGATIVE | NEGATIVE | NEGATIVE | NEGATIVE |
| 168 | 16 WA | NEGATIVE | NEGATIVE | NEGATIVE | NEGATIVE | NEGATIVE |
| 169 | 28 WA | NEGATIVE | NEGATIVE | NEGATIVE | NEGATIVE | NEGATIVE |
| 170 | 40 WA | NEGATIVE | NEGATIVE | NEGATIVE | NEGATIVE | NEGATIVE |
| 171 | 33 WA | NEGATIVE | NEGATIVE | NEGATIVE | NEGATIVE | NEGATIVE |
| 172 | 20 WA | NEGATIVE | NEGATIVE | NEGATIVE | NEGATIVE | NEGATIVE |
| 173 | 39 WA | NEGATIVE | NEGATIVE | NEGATIVE | NEGATIVE | NEGATIVE |
| 174 | 41 WA | NEGATIVE | NEGATIVE | NEGATIVE | NEGATIVE | NEGATIVE |
| 175 | 41 WA | POSITIVE | POSITIVE | POSITIVE | POSITIVE | POSITIVE |
| 176 | 40 WA | POSITIVE | POSITIVE | POSITIVE | POSITIVE | POSITIVE |
| 178 | 38 WA | NEGATIVE | NEGATIVE | NEGATIVE | NEGATIVE | NEGATIVE |
| 180 | 24 WA | NEGATIVE | NEGATIVE | NEGATIVE | NEGATIVE | NEGATIVE |
| 181 | 41 WA | NEGATIVE | NEGATIVE | NEGATIVE | NEGATIVE | NEGATIVE |
| 182 | 40 WA | POSITIVE | POSITIVE | POSITIVE | POSITIVE | POSITIVE |
| 183 | 39 WA | POSITIVE | POSITIVE | POSITIVE | POSITIVE | POSITIVE |
| 184 | 41 WA | POSITIVE | POSITIVE | NEGATIVE | POSITIVE | POSITIVE |
| 185 | 31 WA | NEGATIVE | NEGATIVE | NEGATIVE | NEGATIVE | NEGATIVE |
| 186 | 34 WA | NEGATIVE | NEGATIVE | NEGATIVE | NEGATIVE | NEGATIVE |
| 187 | 33 WA | NEGATIVE | NEGATIVE | NEGATIVE | NEGATIVE | NEGATIVE |
| GM101 | 40 WA | NEGATIVE | NEGATIVE | NEGATIVE | NEGATIVE | NEGATIVE |
| GM102 | 41 WA | POSITIVE | NEGATIVE | NEGATIVE | NEGATIVE | POSITIVE |
| GM103 | 38 WA | NEGATIVE | NEGATIVE | NEGATIVE | NEGATIVE | NEGATIVE |
| GM104 | 37 WA | NEGATIVE | NEGATIVE | NEGATIVE | NEGATIVE | NEGATIVE |
| GM106 | 38 WA | NEGATIVE | NEGATIVE | NEGATIVE | NEGATIVE | NEGATIVE |
| GM107 | 38 WA | NEGATIVE | NEGATIVE | NEGATIVE | NEGATIVE | NEGATIVE |
| GM108 | 23 WA | NEGATIVE | NEGATIVE | NEGATIVE | NEGATIVE | NEGATIVE |
| GM109 | 32 WA | NEGATIVE | NEGATIVE | NEGATIVE | NEGATIVE | NEGATIVE |
| GM111 | 34 WA | NEGATIVE | NEGATIVE | NEGATIVE | NEGATIVE | NEGATIVE |
| GM114 | 41 WA | NEGATIVE | NEGATIVE | NEGATIVE | NEGATIVE | NEGATIVE |
| GM115 | 40 WA | NEGATIVE | NEGATIVE | NEGATIVE | NEGATIVE | NEGATIVE |
| GM116 | 40 WA | NEGATIVE | NEGATIVE | NEGATIVE | NEGATIVE | POSITIVE |
| GM117 | 37 WA | POSITIVE | POSITIVE | POSITIVE | POSITIVE | POSITIVE |
| GM120 | 28 WA | NEGATIVE | NEGATIVE | NEGATIVE | NEGATIVE | NEGATIVE |
| GM121 | 39 WA | POSITIVE | POSITIVE | POSITIVE | POSITIVE | POSITIVE |
| GM122 | 18 WA | POSITIVE | POSITIVE | POSITIVE | POSITIVE | POSITIVE |

Note
WA = weeks of amenorrhea

The clinician's interpretation was given after the end of the pregnancy with knowledge of the entire file, which includes the clinical and ultrasound data at the time the test was carried out, but also the events that subsequently occurred until birth. A diagnosis of rupture was not made when no flow was visible with a speculum despite a cough test, the amount of fluid remained normal on the ultrasound and the patient returned home with no further episode of flow for at least 48 hours or an amniotic sac was clearly seen without clinical flow for patients going into labor within a period of 48 hours. The opinion was rupture when a flow had been indicated and the examination with a speculum with the cough test showed a flow and/or when the amniotic sac was not seen, including during labor in the 48 hours following the test. Patients in whom no flow could be seen, but with an oligohydramnios on the ultrasound, were monitored closely (every 48 hours) or were induced within 48 hours. A diagnosis of rupture was made if the flow categorically recurred within 48 hours or if the amniotic sac was not seen, including during labor for the induced patients.

The veracity of the test could be verified by a complete gynecological examination making it possible to confirm or refute the result.

21.6% of the samples (22 cases out of 102) show a conflict between one of the three markers and the clinical data which represent the opinion of the obstetrician.

Table 2 below describes examples of results of the clinical data and table 3 the clinical data and the interpretation by the clinician.

Table 3: table of examples of results with the clinical data for cases which are not in agreement

TABLE 2
Results of the clinical data

| | | Method of the | Device of | | | |
|---|---|---|---|---|---|---|
| Sample | | state of the art | the invention | | | |
| No. | Term | INTERPRETATION | IGFBP-1 | AFP | INTERPRETATION | Clinical data |
| 119 | 40 WA + 2 d | POSITIVE | NEGATIVE | NEGATIVE | NEGATIVE | NEGATIVE |
| 163 | 32 WA | POSITIVE | NEGATIVE | NEGATIVE | NEGATIVE | NEGATIVE |
| 112 | 40WA + 4 d | POSITIVE | POSITIVE | NEGATIVE | POSITIVE | POSITIVE |
| 126 | 39WA + 2 d | POSITIVE | POSITIVE | NEGATIVE | POSITIVE | POSITIVE |
| 136 | 41WA + 4 d | POSITIVE | POSITIVE | NEGATIVE | POSITIVE | POSITIVE |
| 137 | 37WA + 6 d | POSITIVE | POSITIVE | NEGATIVE | POSITIVE | POSITIVE |
| 184 | 41 WA | POSITIVE | POSITIVE | NEGATIVE | POSITIVE | POSITIVE |

| | | |
|---|---|---|
| Number of false | 2 | 0 |
| positives | | |
| Number of false | 3 | 5 |
| negatives | | |

Note
WA = weeks of amenorrhea
d = days

TABLE 3
Table comprising cases which are not in agreement and the clinical analysis

| | Opinion of | Term | |
|---|---|---|---|
| Sample | the | (weeks of | |
| No. | obstetrician | amenorrhea) | COMMENTS |
| 119 | NEGATIVE | 40 + 2 d | 40 weeks of amenorrhea + 2 days. Spontaneous labor, cervix dilated to |
| | | | 4 cm with amniotic sac visible, losses for 3 days. |
| 163 | NEGATIVE | 32 | 32 weeks of amenorrhea. Suspected losses during vomiting post-OGTT |
| | | | (oral glucose tolerance test). Ultrasound shows normal amniotic fluid |
| | | | and patient leaves hospitalization. No flow for two weeks. |
| 112 | POSITIVE | 40 + 4 d | 40 weeks of amenorrhea + 4 days visible flow. pH does not change. |
| 126 | POSITIVE | 39 + 2 d | 39 weeks of amenorrhea + 2 days, losses indicated, pH does not |
| | | | change. |
| 136 | POSITIVE | 41 + 4 d | 41 weeks of amenorrhea + 4 days. Patient indicates losses. |
| 137 | POSITIVE | 37 | 37 weeks of amenorrhea + 6 days. Patient indicates losses. |
| 115 | POSITIVE | 34 + 4 d | Clear flow at 34 weeks of amenorrhea + 4 days with ultrasound showing |
| | | | diagnosis of oligohydramnios. Patient hospitalized despite a negative |
| | | | ACTIM ® (registered trademark) PROM test since clinical data are so in |
| | | | favor of a rupture of membrane. Continued losses and spontaneous |
| | | | childbirth 4 days later. |
| 184 | POSITIVE | 40 WA + 5 d | Clear rupture, visible flow. |

Note
WA = weeks of amenorrhea
d = days

Table 4 below summarizes the results obtained with the method of the invention compared with the method of the state of the art.

TABLE 4

| | Method of the state of | Method of the invention |
|---|---|---|
| | the art | (IGFBP-1 + AFP) |
| Specificity | 97.10% (67/69) | 100% (64/64) |
| Confidence interval | 89.92-99.65 | 94.4-100 |
| Agreement with the | 95.10% | 94.60% |
| clinical data | | |

Thus, the method of the invention comprising the detection of IGFBP-1 and of AFP makes it possible to obtain a specificity of 100%, thus making it possible to eliminate all false-positive results and to conclude, when AFP and IGFBP-1 are detected, that there is rupture of fetal membranes, contrary to the prior art devices. This is a major improvement since the false-positive results of the prior art tests represent a real problem for the clinician and the patient because they can cause needless medical actions (taking of antibiotics, induced labor or triggering of a cesarean section).

In addition, according to the invention, for the samples for which the results obtained were AFP+/IGFBP-1— (considered to be not determined by the clinician), an additional step of detecting IGFBP-1 with a detection threshold of 5 ng/ml was carried out. Thus, it was possible to increase the sensitivity of the detection of the rupture of fetal membranes as indicated in the following table 5:

TABLE 5

| | | Method of the invention |
|---|---|---|
| | | (IGFBP-1 + AFP) |
| | | comprising an additional |
| | Method of the state of | step of detecting |
| | the art | IGFBP-1 |
| Specificity | 97.10% (67/69) | 100% (69/69) |
| Confidence interval | 89.92-99.65 | 94.79-100 |
| Number of not | 0 | 0 |
| determined | | |
| Agreement with the | 95.10% | 95.10% |
| clinical data | | |

According to another embodiment of the invention, the AFP kinetics were taken into account. In particular, the concentration in the amniotic fluid decreases at the end of pregnancy. Thus, the detection or non-detection of AFP was correlated with the date on which the sample was taken. In particular, the sample was classified according to whether it was taken before or during/after the 39^{th} week of amenorrhea.

Table 6 below summarizes the results obtained and the date of sampling

TABLE 6

| | False negatives for | False positives for |
|---|---|---|
| | AFP | AFP |
| Population of which the term is | 0 | 5 |
| less than 39 WA | | |
| Population for which the term is | 5 | 0 |
| greater than or equal to 39 WA | | |

As previously demonstrated, in this embodiment, the rupture is detected with the method of the invention with certainty.

In addition, according to this embodiment, the population tested for whom the test was AFP/IGFBP-1 was divided in half depending on whether the sample came from patients whose term was less than 39 weeks (subgroup 1) or from patients whose term was greater than or equal to 39 weeks (subgroup 2). In the present example, subgroup 1 corresponds to 45 patients among whom no false positive was demonstrated, and subgroup 2 corresponds to 24 patients in whom 5 false negatives are detected. The population tested for whom the test was AFP+/IGFBP-1— was divided according to whether the sample came from patients whose term was less than 39 weeks (subgroup 3) or from patients whose term was greater than or equal to 39 weeks (subgroup 4).

As demonstrated in table 7 below, the method of the invention makes it possible, when excluding the patients corresponding to subgroups 2 and 3, to detect the rupture or non-rupture of fetal membranes with a sensitivity and a specificity of 100%. Conversely, the methods of the state of the art do not make it possible to obtain such results. In particular, false positives and false negatives are obtained with the known devices, without it being possible to identify subgroups of patients in whom the result is 100% reliable.

TABLE 7

| | Method of the state of | Method of the invention |
|---|---|---|
| | the art | (IGFBP-1 + AFP) |
| Performance levels | 3 false negatives, | 0 false positive, |
| | 2 false positives | 0 false negative |
| Sensitivity | 90.91% (30/33) | 100% (24/24) |
| Confidence interval | 75.67-98.08 | 85.75-100 |
| Specificity | 97.10% (67/69) | 100% (48/48) |
| Confidence interval | 89.92-99.65 | 92.60-100 |
| Number of cases not | 0 | 30/102 (29.4%) |
| determined | | |
| Agreement with the | 95.10% | 100.00% |
| clinical data | | |

As demonstrated in this example, the method of embodiments of the invention makes it possible to obtain a reliable result of rupture or non-rupture of fetal membranes, making it possible to dispense with supplementary tests and/or clinical examinations.

### LIST OF REFERENCES

1. Rutanen et al. Radioimmunoassay of placenta protein 12: levels in amniotic fluid, cord blood and serum of healthy adults, pregnant women and patients with trophoblastic disease. Am. J. Obstet. Gynecol. 1982
2. Rochelson et al. Rapid assay: possible application in the diagnosis of premature rupture of the membranes. Obstet Gynecol 1983; 62:414-418
3. Young-Han Kim et al. Vaginal fluid b-human chorionic gonadotropin level in the diagnosis of premature rupture of membranes. Acta Obstet Gynecol Scand 2005: 84: 802-805
4. Koninckx P R et al. Prolactin concentration in vaginal fluid: a new method for diagnosing ruptured membranes. Br J Obstet Gynaecol 1981; 88:607-610
5. Rutanen et al, “Monoclonal antibodies to the 27-34K insulin-like growth factor binding protein” Biochem Biophys Res Commun 1988; 152: 208
6. Pekonen et al, “A monoclonal antibody-based immunoradiometric assay for low molecular weight insulin-like growth factor binding protein/placental protein 12”, J immunoassay 1989; 10: 325-337
7. Beer et al. Qualification of cellulose nitrate membranes for lateral-flow assays, IVD technology, January 2002.
8. Anne Harwood Peruski et al. Methods for Detection and Identification of Infectious Disease and Biological Warfare Agents. Clinical and diagnostic laboratory immunology, July 2003, p. 506-513
9. Rutanen et al. Measurement of Insulin-like growth factor binding protein-1 in cervical/vaginal secretions: comparison with the ROM-Check Membrane Immunoassay in the diagnosis of ruptured fetal membranes. Clinica Chimica Acta, 214 (1993), 73-81
10. Rutanen et al. Decidual transformation of human extrauterine mesenchymal cells is associated with the appearance of insulin like growth factor binding protein 1. J. Clin. Endocrinol. Metab. 1992; 72: 27-31
11. Rutanen et al. Evaluation of a rapid strip test for insulin-like growth factor binding protein 1 in the diagnosis of foetal ruptured membranes. Clinica Chimica Acta. 253; (1996) 91-101
12. Kishida et al. Diagnosis of premature rupture of the membranes in preterm patients, using an improved AFP kit: comparison with ROM-Check and/or nitrazine test. European Journal of Obstetrics and Gynecology and Reproductive Biology 69 (1996) 77-82
13. Bell C. Secretory endometrial and decidual proteins: studies and clinical significance of a maternally derived group of pregnancy associated serum proteins. Human Reproduction. Vol. 1-3 129-143. 1986.
14. Nazimova et al. Blood Serum Content of PAMG-1 Protein Binding Insulin like Growth Factor 1 (Somatomedin C) in Patients with Diabetes Mellitus. Bullet. Experim. Biol. and Med. 166/9 September 1993

1. A method of in vitro detection of a rupture of fetal membranes comprising:
simultaneously searching, in a specimen of vaginal or cervical secretions, for alpha-fetoprotein (AFP) and for insulin-like growth factor-binding protein 1 (IGFBP-1).

2. The method according to claim 1, wherein the search is carried out with at least one capture antibody directed against AFP and with at least one capture antibody directed against IGFBP-1.

3. The method as according to claim 1, wherein the search is carried out using a specimen of vaginal or cervical secretions which is diluted in a buffer solution or medium.

4. The method according to claim 3, wherein the method comprises an adjustment of at least one of parameters chosen from a volume, a pH, and an ionic strength of the buffer medium, said adjustment being common to AFP and to IGFBP-1.

5. The method according to claim 3, wherein the method comprises an adjustment of at least one of parameters chosen from a concentration of capture antibodies and/or a concentration of labeled antibodies, said adjustment being specific to AFP or to IGFBP-1.

6. The method according to claim 3, wherein a pH of the buffer solution is from 5 to 10.

7. The method according to claim 3, wherein anti-AFP and anti-IGFBP-1 capture antibodies are in dry form.

8. The method according to claim 1, wherein said searching step comprises a visualizing step carried out with at least one anti-AFP labeled antibody and at least one anti-IGFBP-1 labeled antibody.

9. The method according to claim 1, wherein an additional step of detecting IGFBP-1 alone is carried out.

10. The method as claimed in claim 9, wherein said IGFBP-1 is detected starting from a concentration of from 1 to 10 ng/ml.

11. A device for implementing the method according to claim 1, the device comprising:
a zone for depositing a sample;
a zone comprising labeled anti-IGFBP-1 antibodies and labeled anti-AFP antibodies;
a visualizing zone comprising capture antibodies directed against AFP and capture antibodies directed against IGFBP-1.

12. The device according to claim 11, wherein the depositing zone is either an absorbent paper or a glass fiber support.

13. The device according to claim 11, wherein the visualizing zone is a nitrocellulose membrane.

14. The device according to claim 11, wherein the zone comprising labeled anti-IGFBP-1 antibodies and labeled anti-AFP antibodies is a glass fiber support.
